(19) United States
(12) Patent Application Publication
(10) Pub. No.: US 2014/0350515 A1
(43) Pub. Date: Nov. 27, 2014

(54) Ultra Flexible Surgical Drain and Catheter

(71) Applicants: Cyrus Omid Abbasi, Albuquerque, NM (US); Bret Baack, Albuquerque, NM (US); Tariq A. Khraishi, Albuquerque, NM (US)

(72) Inventors: Cyrus Omid Abbasi, Albuquerque, NM (US); Bret Baack, Albuquerque, NM (US); Tariq A. Khraishi, Albuquerque, NM (US)

(21) Appl. No.: 14/284,140

(22) Filed: May 21, 2014

Related U.S. Application Data
(60) Provisional application No. 61/825,943, filed on May 21, 2013.

(51) Int. Cl. A61M 25/00; A61M 27/00

(52) U.S. Cl. CPC A61M 25/0054; A61M 27/00; A61M 2210/1021
USPC 604/506; 604/525

(57) ABSTRACT

A new flexible surgical drain/catheter system is provided. The flexible system encompasses a flexible section that can be incorporated in the design of surgical drains/catheters of any kind. By incorporating this flexible system, a surgical. drain/catheter will possess three significant additional characteristics. These additional characteristics will make the herein designs superior to the existing designs. The first characteristic is that the surgical drain/catheter can be bent without causing any fold/crease to its outer and inner surfaces. The second characteristic prevents internal occlusions while the drain/catheter is bent. The third characteristic is that the flexible section can also be used as a reliable region of drain/catheter anchorage to the patient's body.

### CROSS-REFERENCE TO RELATED APPLICATIONS

This application claims the benefit U.S. Provisional Application No. 61/825943 filed May 21, 2013 and herein incorporated by reference.

### STATEMENT REGARDING FEDERALLY SPONSORED RESEARCH OR DEVELOPMENT

Not Applicable.

INCORPORATION-BY-REFERENCE OF MATERIAL SUBMITTED ON A COMPACT DISC

Not Applicable.

### BACKGROUND OF THE INVENTION

1. Field of the Invention

Surgical drains and catheters are efficient and reliable means to extract or relocate undesirable bodily fluids from or within the body of a patient during and after a surgery. Surgical drains and catheters have been circular-bodied or rectangular-bodied. The shape of the device is typically chosen by the surgeon according to the type of surgical procedure being conducted as well as the location and placement of the device inside or outside of a patient's body.

For instance, after the removal of an abdominal cancerous tumor, the remaining cavity is usually tilled with bodily fluids such as blood that can cause infections and delay the healing process of the affected area. Usually for an abdominal surgical procedure, circular devices are utilized. After employing the catheter within or extending out of the body, they are connected to vacuum chambers, which extract the undesirable fluids via the suction forces induced by the vacuum chambers.

The catheters are held in place by suture-threads that are knotted around the outer surface of the catheter and then sutured to the skin or the surrounding tissue. Then the remainder of the extruding drain is folded and taped flush against the skin of the patient. This is the most widely used catheter-to-the-body attachment technique.

Several problems arise after using this mode of attachment. Upon constant respiration of the patient, the knots involved in the anchoring sutures might loosen and the catheter can move and be displaced from its intended position. In situations where the catheter is attached to the skin of a patient, it has been reported that the natural respiratory motion of the patient may cause knots to loosen within a period of 2 to 3 weeks. Furthermore, it has also been reported that upon accidental events (e.g. such as the catheter getting stuck to a doorknob) the entire device can be pulled out due to the rupture of the sutures or the anchoring skin/tissue. Hence, a second surgery will be needed to place a new drain/catheter.

Another problematic pattern is developed when the catheter is folded and taped along the skin. The folding of the catheter causes internal occlusion in the lumen of the device that leads to reduced fluid drainage. Moreover, folding induces creases along the internal and external surfaces of the catheter, which may lead to internal and external damage to the catheter.

Another problem is faced in sonic cases of chest and abdominal trauma. When a patient has severe internal bleeding, the use of large diameter drains (0.75 cm to 2.0 cm external diameter) is routine. The high diameter drain is usually run through the ribs of the patient connecting the internal and external environments of the patient's body. The high diameter drains are highly inflexible and maintain an almost perpendicular position to the patient's skin. En other words, after they exit the body through the ribs, the device protrudes outwardly and cannot be made flush with the skin unless brute force is used. This forcing action not only induces severe internal drain occlusions but also causes tensile and compressive forces in the skin/tissue, which in turn cause severe pain to the patient.

Moreover, the above-described conventional catheter-to-the-body attachment becomes more and more unreliable as the diameter of the catheter increases. This is because as the diameter of the drain increases, more suture material is needed to be wrapped around the drain to hold it in place. The longer the sutures, the higher the probability of the suture's slippage and their tangling.

### BRIEF SUMMARY OF THE INVENTION

In one aspect, a preferred embodiment of the present invention provides a new drain and/or catheter having flexible sections that act as segments around which sutures can be securely wrapped. The rims and grooves of the flexible section provide the perfect geometry within which sutures can securely fit without any subsequent slippage. The sutures can then be run through the skin or any other underlying tissue to ensure a secure mode of attachment. It should be noted that the herein-described flexible sections can be incorporated in lost if not all of the conventional surgical drains and/or catheters, namely devices without the flexible section. In other aspects, the devices may be manufactured from biocompatible and flexible polymer material.

### BRIEF DESCRIPTION OF THE SEVERAL VIEWS OF THE DRAWINGS

FIG. 1 is a right view of one embodiment of the invention having rims and grooves having semicircular edges.

FIG. 2 is a cross-sectional the embodiment show in FIG. 1 at a bend of 90 degrees.

FIG. 3 front view of the embodiment shown in FIG. 1 at a bend of 180 degrees.

FIG. 4 is an isometric view of the embodiment shown in FIG. 1 illustrating the use of sutures to affix the device.

FIG. 5 is a right view of one embodiment the invention having rims at and grooves having triangular edges.

FIG. 6 is a cross-sectional of the embodiment shown in FIG. 5 at a bend of 90 degrees.

FIG. 7 is a right view of one embodiment of the invention having rims and grooves having rectangular edges.

FIG. 8 is a cross sectional of the embodiment shown in FIG. 7 at a bend of 90 degrees.

FIG. 9 is an isometric view of an embodiment having rectangular body.

### DETAILED DESCRIPTION OF INVENTION

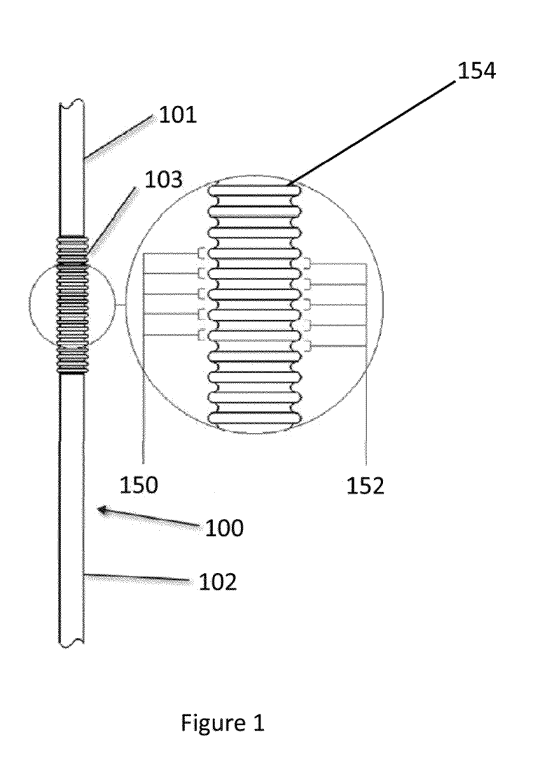

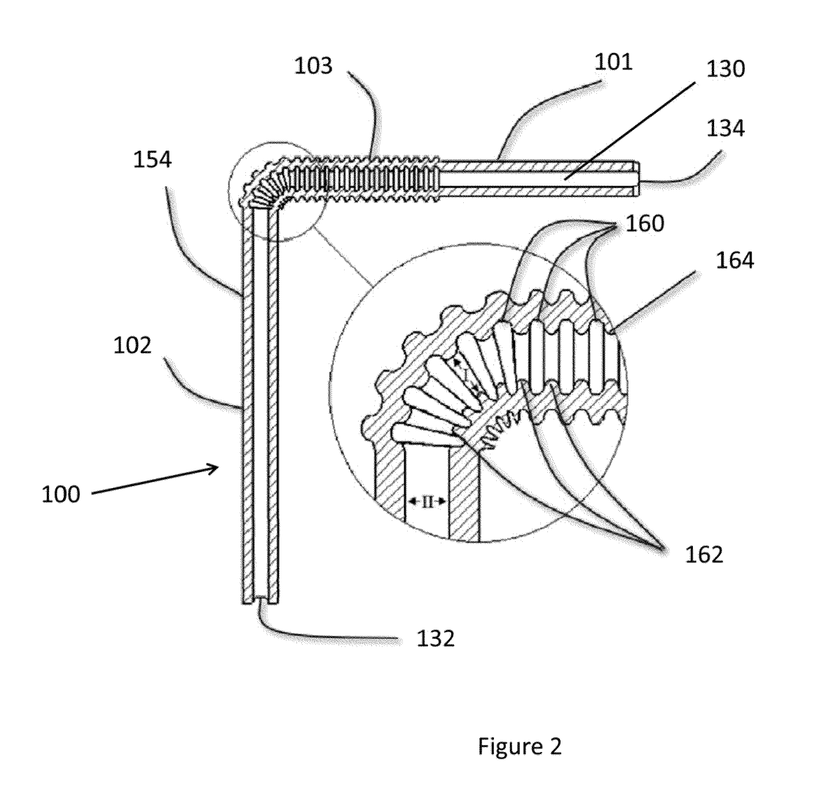

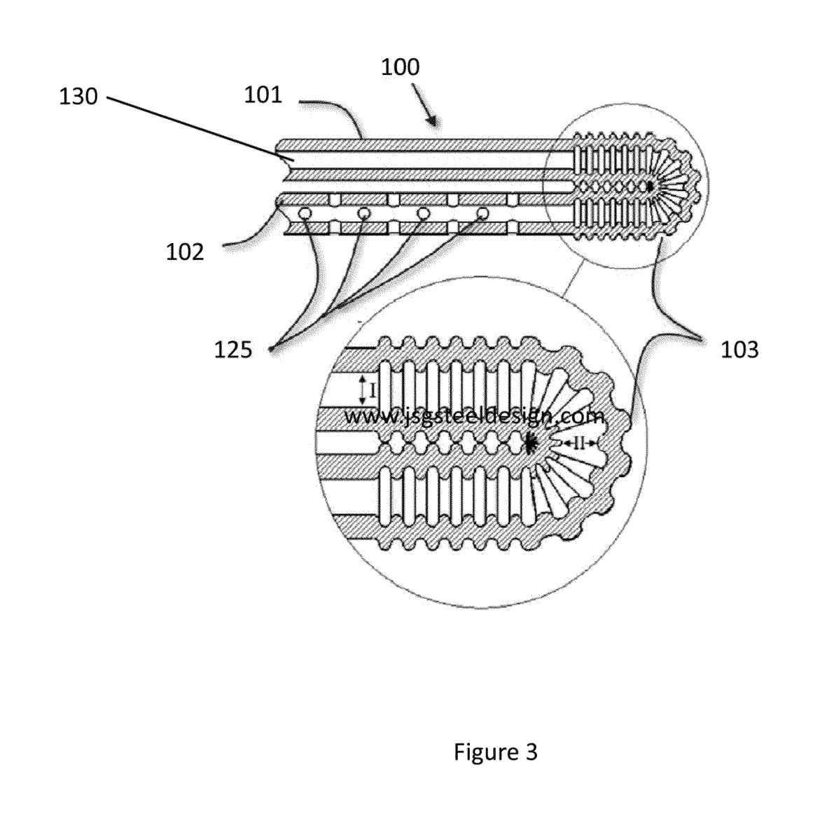

This description is not to be taken its a limiting sense, but is made merely for the purpose of illustrating the general principles of the invention. The scope of the invention is defined by the appended claims. In a preferred embodiment, as shown in FIGS. 1 through 4, the drain/catheter 100 of one preferred embodiment is comprised of a tube that may have three segments comprised of sections 101 and 102 with a flexible section 103 disposed in between. As shown in FIG. 2. catheter 100 has a lumen 130 that runs from distal end 132 to proximal end 134. As show in FIG. 3, lumen 130 may have perforations 125 that are in communication with the lumen to assist in the intake of fluids.

As shown in FIGS. 1 and 2, section 103 includes a plurality of rims grooves 152 on outer wall 154 and a plurality of rims 160 and grooves 162 on inner wall 164. The rims and grooves stretch apart and compress together as shown in FIGS. 2 and 3 so as to permit the device to have the ability to be sharply bent. As shown, the rims at groves on the outer side of the bend stretch apart and the rims and grooves on the inner side of the bend co p together. As is also shown, the internal diameter of lumen 130 in section 103 remains nearly constant for any bend. Specifically, internal diameters “I” and “II” of lumen 130 remain equal or equal or equal in bends up to 90 degrees and in bends up to 180 degrees.

The above-mentioned increased flexibility prevents the formation of internal and external transverse folds and creases when the device is bent or folded into position. Without an internal or external fold or crease, the lumen of the device does not close or collapse and maintains its initial diameter, i.e., the internal diameter of lumen 130. By doing so, the flow of fluids within the lumen is not hindered and stays continuous at the same as within the straight portion of the drain/catheter.

When a surgeon applies a sharp bend, i.e. a bend with a relatively low radius of curvature to a catheter that incorporates section 103, no transverse folds or creases will be induced to the cross-section of the device even if the device undergoes a 180-degree bend. This means that the lumen remains fully open and no hindrance is introduced to any fluid flow within the lumen.

As shown, there is also no expansion or collapse of the external diameter of outer wall 154 along any of the transverse directions. This means that the device does not expand into its surrounding space once it is bent.

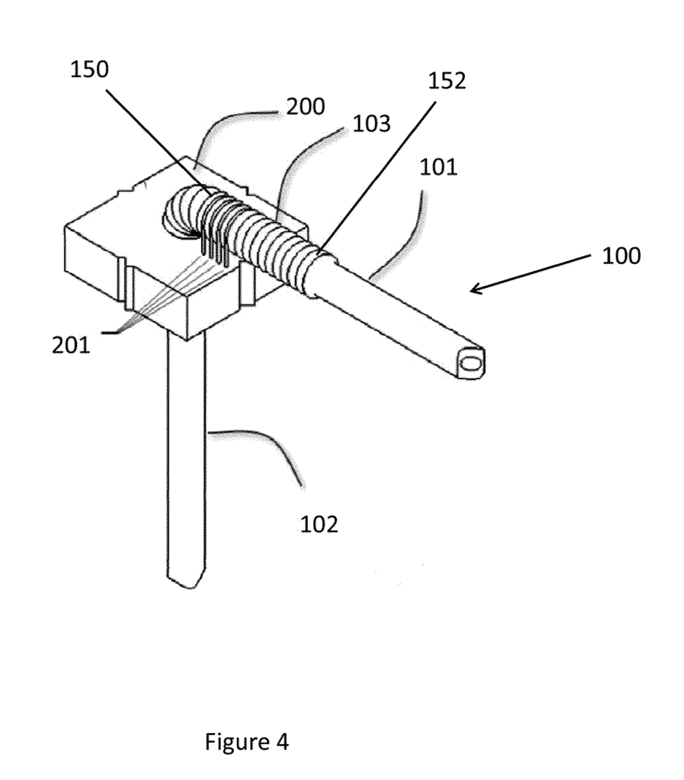

Section 103 can also be utilized as a suitable and reliable point of the catheter anchorage to the underlying skin/tissue. FIG. 4 illustrates how catheter 100 can be securely attached to the underlying skin/tissue. The same kind of attachment can be applied to other geometries as well. As illustrated, a major portion of section 102 is within the body of the patient and comprises a smooth outer wall for ease of use. The upper part of section 102, namely its connecting part to section 103, penetrates and is surrounded by the skin/tissue of the patient. As seen, the bend occurs within section 103 at the junction where the main tube transitions from a smooth surface to bendable section 103 at the skin surface. Locating section adjacent to the patient avoids having to locate the rims and grooves inside the tissue.

Depending on the geometry of the underlying skin/tissue, the angle of the bend can vary from 0 to 180 degrees. Note the bend occurs within the z-y plane. However, the bend can be made within other planes that are parallel to the z-axis and also pass through it. Bends with an angle greater than 180 degrees are also possible if the geometry/space of the underlying skin/tissue allows it. After the bend terminates, the remaining and straight portion of section 103 can be used for additional bends and/or anchorage purposes.

A safe anchorage to the underlying skin/tissue 200 is made by utilizing surgical sutures 201. As shown in FIG. 4, the sutures pass through the underlying skin/tissue and then wrap around the drain/catheter by fitting within the external grooves 152 of section 103. Once the sutures are in place and tightened, they can no longer slip along the surface. This is because rims 150 and grooves 152 of act as physical barriers, which prevent dislodgement of the sutures.

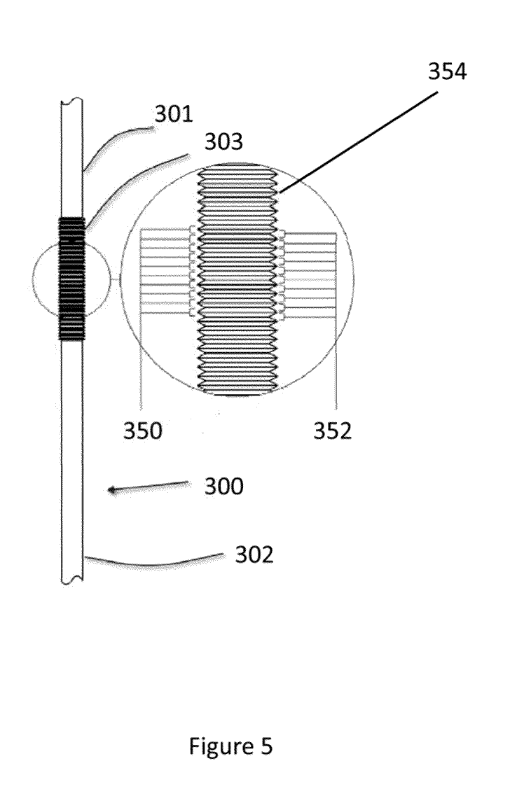

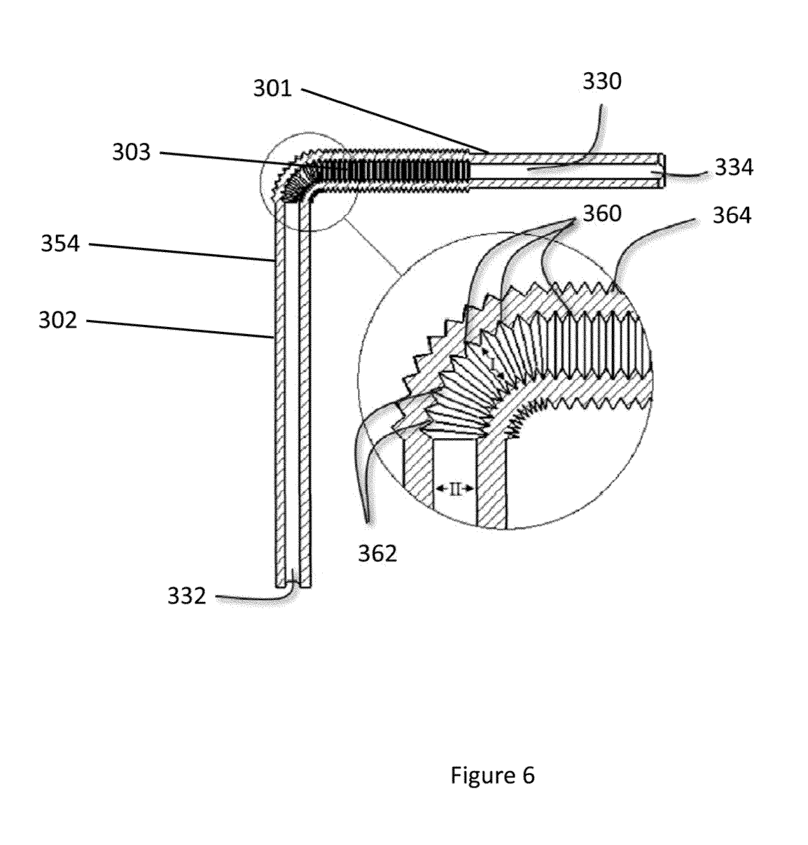

A number of different geometries may be used for the edges of the rims and grooves including, but not limited to semicircular, triangular and rectangular. A semicircular configuration is shown in FIGS. 1 through 4. FIGS. 5 and 6 represent a catheter 300 with sections 301 and 302 as well as a flexible section 303. As illustrated, section 303 provides a plurality of rims 350 and grooves 352 on outer wall 354 and a plurality of rims 360 and grooves 362 on inner wall 364. The edges of the rims and grooves for this embodiment are triangular. All the triangles defined by the rims 350 and grooves 352 may be equilateral triangles.

A shown in FIG. 6, “I” represents the shortest internal diameter within section 303, namely the distance between the internal rims. “II” represents the diameter of the internal passage of lumen 330 of catheter 300. As shown, the external triangular rims do not extend much into the surrounding space around catheter 300. Therefore, catheter 300 can be placed through natural or incised openings with diameters that are very close to the external diameter of the device. Therefore, no occlusions are introduced within the internal passage of catheter 300. Hence, the flow of fluids within catheter 300, which incorporates a flexible section 303, is not hindered by internal occlusions.

Analogous to the anchoring method described above, surgical sutures pass through the underlying skin/tissue of the patient and then wrap around section 303 with the rims and grooves preventing any motion of the catheter unit along the y-axis. Moreover, other movements of the drain/catheter unit are prevented as well.

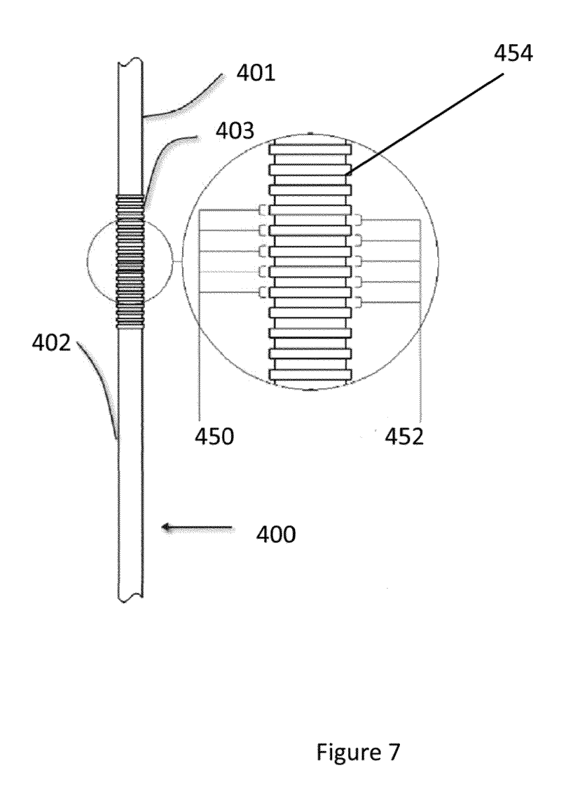

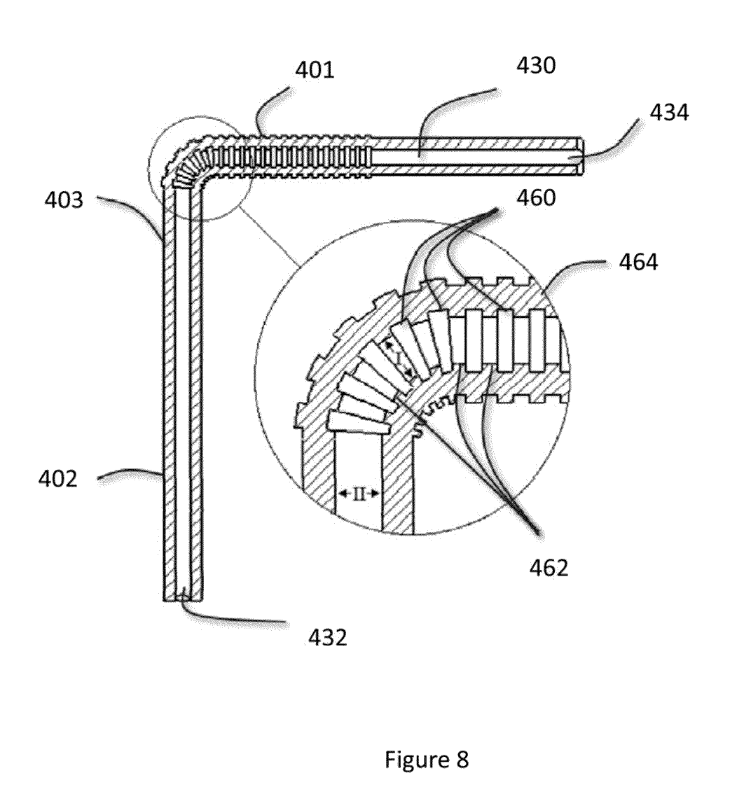

FIGS. 7 and 8 depict a catheter 400 having sections 401 and 402 as well as a flexible section 403. As illustrated, section 403 provides a plurality of rims 450 and grooves 452 on outer wall 454 and a plurality of rims 460 and grooves 462 on inner wall 464. The edges of rims and groves are rectangular in shape.

As shown in FIG. 8, “I” represents the shortest internal diameter within section 403, namely the distance between the internal rims. “II” represents the diameter of the internal passage of lumen 430 of catheter 400. As shown, the external rectangular rims 450 do not extend much into the surrounding space around catheter 400. Therefore, the catheter can be placed through natural or incised openings with diameters that are very close to the external diameter of sections 401 and 402. Therefore, no occlusions are introduced within the internal passage of the catheter. Hence, the flow of fluids within a catheter, which incorporates a flexible section 403, is not hindered by internal occlusions.

Analogous to the anchoring, method described above, surgical sutures pass through the underlying skin/tissue of the patient and then wrap around section 403 with the rims and grooves preventing any motion of the drain/catheter unit along the y-axis. Moreover, other movements of the drain/catheter unit are prevented as well.

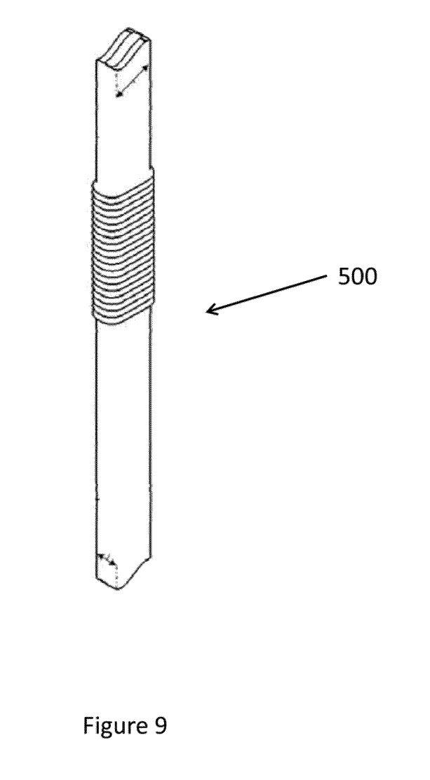

FIG. 9 illustrates a rectangular drain/catheter 500 that may use the semicircular, triangular and rectangular rim and groove configurations described above. Moreover, for the embodiments described above, the flexible sections impart a three-dimensional bendability to the device with almost infinite degrees of rotational freedom. This flexibility is maintained without 1) inducing any occlusion or tightening of the lumen of the bent section; 2) inducing any transverse folds or creases in the flexible section; 3) inducing any expansion or collapse of the lumen within the flexible section; 4) inducing any change in the external and internal diameter of the flexible section; 5) inducing any strain/stress to the underlying/surrounding skin/tissue of the patient's body; and 6) inducing any unnecessary pain to patient caused by the bending of the device.

The three-dimensional flexibility also dampens the longitudinal forces involved in any natural or accidental pulling action on the device. The pulling action can he internal or external to a patient's body and reduces the risk of accidental dislodgment. The three-dimensional flexibility reduces the risk of accidental removal from to patient's body. The three-dimensional flexibility reduces the risk of a drain's/catheter's accidental slippage and with that the need of a secondary operation to replace the drain/catheter at its intended position.

The rims and grooves described above enable a drain/catheter to be securely attached to its underlying/surrounding skin/tissue. They also allow surgical sutures to nest within the grooves for retaining the device by acting as physical barriers that restrict the slippage of the surgical sutures along the external surface of the device. In addition, one or more flexible sections may be provided in a single device. In addition, other cross-sectional geometries may be used as well such a square, rectangular, oblong, triangular and other known shapes for the shape of the overall device.

A method of securing a catheter to a patient using the embodiments described above includes inserting a portion of the catheter into a patient wherein, as set forth above, the catheter comprises a main tube comprising a distal end, a proximal end, and outer wall having an outer diameter and an inner wall defining a lumen having an inner diameter that extends from a distal end to a proximal end. The main tube includes at least one bendable section connected to a smooth tube section. The bendable section has a first set of a plurality of rims and grooves on an outer wall and a second set of a plurality of rims and grooves on an inner all that forms a lumen. The first and second sets of rims and grooves are adapted to stretch and compress when the bendable section is bent from a straight position to an angled position and the inner and outer diameters of the bendable section remain constant when the bendable section is bent. The smooth portion of the main tube is positioned in the patient and the bendable section is positioned adjacent to patient's skin or the tissue surface. This locates the bendable section in a position in which the bendable section is substantially parallel to the skin surface of the patient. The catheter is then sutured in place by wrapping sutures around the bendable section with the sutures located in one or more of the grooves on the outer wall to secure the catheter in place.

What is claimed is:

1. A bendable catheter comprising:
a main tube comprising a distal end, a proximal end, an outer wall and an inner wall defining a lumen having a diameter that extends from said distal end to said proximal end;
said main tube including at least one bendable section, said bendable section having a first set of a plurality of rims and grooves on said outer wall and a second set of a plurality of rims and grooves on said inner wall forming a lumen;
said rims and grooves adapted to stretch and compress when said bendable section is bent from a straight position to an angled position; and
said diameter of said lumen at said bendable section remains constant when said bendable section is bent.

2. The device of claim 1 wherein said diameter of said lumen of said bendable section remains constant when said bendable section is bent at an angle up to 90 degrees.

3. The device of claim 1 wherein said diameter of said lumen of said bendable section remains constant when said bendable section is bent at an angle up to 180 degrees.

4. The device of claim 1 wherein said plurality of rims and grooves have edges that are semicircular.

5. The device of claim 1 wherein said plurality of rims and grooves have edges that are triangular.

6. The device of claim 1 wherein said plurality of rims and grooves have edges that are rectangular.

7. The device of claim 1 wherein said grooves of said first set of rims and groves are adapted to receive a suture.

8. A method of securing a catheter to a patient having a tissue surface comprising:
inserting a portion of said catheter into a patient;
said catheter comprising: a main tube comprising, a distal end, a proximal end, and outer wall having an outer diameter and an inner wall defining a lumen having an inner diameter that extends from said distal end to said proximal end;
said main tube including at least one bendable section connected to said smooth tube section, said bendable section having a first set of a plurality of rims and grooves on said outer wall and a second set of a plurality of rims and grooves on said inner wall forming a lumen;
said rims and grooves adapted to stretch and compress when said bendable section is bent from a straight position to an angled position;
said inner and critter diameters at said bendable section remain constant when said bendable section is bent;
positioning said smooth tube section in said patient and said bendable section in a position adjacent to said patient tissue surface;
bending said bendable section into a position in which said bendable section is substantially parallel to said patient tissue surface; and
suturing said catheter to said patient by wrapping sutures around said bendable section and said sutures located in one or more grooves on said outer wall.

9. The method of claim 8 wherein said inner and outer diameters at said bendable section remain constant when said bendable section is bent at an angle up to 90 degrees.

10. The method of claim 8 wherein said inner and outer diameters at said bendable section remain constant when said bendable section is bent at an angle up to 80 degrees.

11. The method of claim 8 wherein said plurality of rims and grooves have edges that are semicircular.

12. The method of claim 8 wherein said plurality of rims and grooves have edges that are triangular.

13. The method of claim 8 wherein said plurality of rims and grooves have edges that are rectangular.

14. The method of claim 8 wherein said grooves of said first set of rims and groves are adapted to receive a suture.
